(19) United States
(12) Patent Application Publication
(10) Pub. No.: US 2008/0058511 A1
(43) Pub. Date: Mar. 6, 2008

(54) METHODS AND COMPOSITIONS USEFUL IN THE PREPARATION OF OLIGONUCLEOTIDES

(76) Inventors: JOHN HARGREAVES, Mountain view, CA (US); Eric M. Leproust, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/469,398

(22) Filed: Aug. 31, 2006

(51) Int. Cl. C07H 21/04; C07H 21/00

(52) U.S. Cl. 536/25.3

(57) ABSTRACT

Provided are improved processes for solid phase oligonucleotide synthesis, the improvements comprising carrying out detritylation of the nascent oligonucleotide using a composition of an organic solvent, a protic solvent and a protic acid. In some embodiments, a protic solvent is present at a concentration sufficient to destroy one or more impurities that can act as capping agents during a detritylation step and which reduce the oligonucleotide yield. Exemplary protic solvents include water, alcohols, or amines. Exemplary protic acids include monochloroacetic acid, dichloroacetic acid, and trichloroacetic acid.

### FIELD

The disclosure relates to the chemical synthesis of oligonucleotides and to materials and processes that are useful in such synthesis.

### BACKGROUND

Oligonucleotides have become indispensable tools in modern molecular biology, being used in a wide variety of techniques, ranging from diagnostic probing methods to PCR to antisense inhibition of gene expression. This widespread use of oligonucleotides has led to an increasing demand for rapid, inexpensive and efficient methods for synthesizing oligonucleotides. The synthesis of oligonucleotides for antisense and diagnostic applications can now be routinely accomplished (see, e.g., Methods in Molecular Biology. Vol 20: Protocols for Oligonucleotides and Analogs pp. 165-189 (S. Agrawal, Ed., Humana Press, 1993); Oligonucleotides and Analogues: A Practical Approach pp. 87-108 (F. Eckstein, Ed., 1991); Agrawal and Iyer (1995) Curr. Op. in Biotech. 6:12; Antisense Research and Applications (Crooke and Lebleu, Eds., CRC Press, Boca Raton, 1993); Caruthers (1985) Science 230:281-285; Itakura et al., Ann. Rev. Biochem. 53: 323-356; Hunkapillar et al. (1984) Nature 310:105-110; and in “Synthesis of Oligonucleotide Derivatives in Design and Targeted Reaction of Oligonucleotide Derivatives (CRC Press, Boca Raton, Fla.) pages 100 et seq.; U.S. Pat. No. 4,458,066; U.S. Pat. No. 4,500,707; U.S. Pat. No. 5,153,319; U.S. Pat. No. 5,869,643; EP 0294196). Early synthetic approaches included phosphodiester and phosphotriester chemistries. Khorana et al., J. Molec. Biol. 72: 209 (1972) discloses phosphodiester chemistry for oligonucleotide synthesis. Reese (1978) Tetrahedron Lett. 34:3143-3179, discloses phosphotriester chemistry for synthesis of oligonucleotides and polynucleotides. These early approaches have largely given way to the more efficient phosphoramidite and H-phosphonate approaches to synthesis. Beaucage and Caruthers (1981) Tetrahedron Lett. 22:1859-1862, discloses the use of deoxynucleoside phosphoramidites in polynucleotide synthesis. Agrawal and Zamecnik, U.S. Pat. No. 5,149,798 (1992), discloses optimized synthesis of oligonucleotides by the H-phosphonate approach.

Machines for the automated synthesis of support-bound single stranded DNA have been described (see, e.g., Matteucci and Caruthers (1981) J. Amer. Chem. Soc. 103:3185-3191; and Gait, ed., Oligonucleotide Synthesis: A Practical Approach (IRL Press, Washington, D.C., 1984)). A synthetic cycle is repeated under computer control to add one nucleoside monomer unit at a time to achieve the desired sequence and length which defines the oligonucleotide. For example, within the phosphoramidite, or phosphite triester, synthetic cycle several reactions can be used:

I. Deprotect the reactive functionality (usually a 5′ hydroxyl) on the growing chain;

II. Achieve coupling by the addition of a monomer and activator;

Ill. Cap unreacted 5′ hydroxyls to prevent further coupling to failure sequences;

IV. Oxidize the newly formed internucleotide phosphorous linkage to the naturally occurring pentacoordinate state; and

V. Optionally cap unreacted 5′ hydroxyls to prevent further coupling to failure sequences and to remove water introduced by the oxidation reaction.

In some applications, an oligonucleotide is synthesized on a solid support such as in an array. Oligonucleotide arrays (such as DNA or RNA arrays), are known and are used, for example, as diagnostic or screening tools. Such arrays include regions of usually different sequence oligonucleotides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon interrogating the array. For example all polynucleotide targets (for example, DNA) in the sample can be labeled with a suitable label (such as a fluorescent compound), and the fluorescence pattern on the array accurately observed following exposure to the sample. Assuming that the different sequence oligonucleotides were correctly deposited in accordance with the predetermined configuration, then the observed binding pattern will be indicative of the presence and/or concentration of one or more polynucleotide components of the sample.

Oligonucleotide arrays can be fabricated using in situ synthesis methods (see, e.g., WO 95/25116 and WO 98/41531, and the references cited therein). The in situ method for fabricating an oligonucleotide array typically follows, at each of the multiple different addresses at which features are to be formed, the same conventional sequence used in forming oligonucleotides on a support by means of known chemical processes as described above.

### SUMMARY

In some embodiments, the present disclosure provides improved processes for solid phase oligonucleotide synthesis. In these improved processes, the improvement comprises carrying out detritylation of the nascent oligonucleotide using a composition of an organic solvent, a protic solvent and a protic acid. In some embodiments, the composition comprises an organic solvent, a protic acid, and a protic solvent present at a concentration greater than about 0.0575% mole fraction. In some embodiments, the protic solvent comprises at least one of water, alcohols, and amines. Exemplary protic acids include monochloroacetic acid, dichloroacetic acid and trichloroacetic acid. In some embodiments, the concentration of the protic acid is in the range from about 0.05% mole fraction to about 25.0% mole fraction. In some embodiments, the concentration of the protic acid is in the range from 0.625% mole fraction to 25.0% mole fraction.

In some embodiments, there are provided improved processes for solid phase polynucleotide synthesis, the improvement comprising detritylating the nascent polynucleotide having one or more trityl group in a composition comprising an organic solvent, a protic acid, and a protic solvent, wherein the protic solvent is present at a concentration sufficient to substantially destroy an impurity comprising a capping agent.

In some embodiments, there are provided improved processes for solid phase polynucleotide synthesis, the improvement comprising detritylating the nascent polynucleotide having one or more trityl group in a composition comprising an organic solvent, a protic acid, and a protic solvent, wherein the protic acid is capable of forming a capping agent, and wherein the protic solvent is present at a concentration sufficient to substantially inhibit the formation of said capping agent.

In some embodiments, such synthesis is carried out using the phosphoramidite, H-phosphonate, or phosphotriester approach.

In some embodiments, the present disclosure provides compositions useful in carrying out detritylation of a nascent oligonucleotide during solid phase oligonucleotide synthesis.

Processes and compositions for synthesizing oligonucleotides as disclosed herein are useful for synthesizing oligonucleotides on a scale ranging from small laboratory scale to large commercial scale. Thus, the processes can be used to supply oligonucleotides for research purposes, for diagnostic purposes and for therapeutic purposes.

### BRIEF DESCRIPTION OF THE DRAWINGS

FIG. 1 shows the structure of dichloroacetic anhydride (DCAA).

FIG. 2 shows an embodiment of recapping of a hydroxyl group by DCAA.

FIG. 3 illustrates hydrolysis of DCAA by an exemplary protic solvent.

FIG. 4 shows HPLC traces of oligonucleotides chemically synthesized utilizing different detritylation solutions.

FIG. 5 shows HPLC traces of chemically synthesized oligonucleotides and illustrating the effect of a protic solvent added to a detritylation solution.

FIG. 6 illustrates the effect of an exemplary protic solvent in a detritylation solution on the yield of chemically synthesized oligonucleotides.

### DETAILED DESCRIPTION

Before describing the present disclosure in detail, it is to be understood that this disclosure is not limited to specific compositions, method steps, or equipment, as such can vary. It is also to be understood that the terminology used herein is for the purpose of describing particular embodiments only, and is not intended to be limiting. Methods recited herein can be carried out in any order of the recited events that is logically possible, as well as the recited order of events. Furthermore, where a range of values is provided, it is understood that every intervening value, between the upper and lower limit of that range and any other stated or intervening value in that stated range is encompassed within the present disclosure. Also, it is contemplated that any optional feature of the inventive variations described can be set forth and claimed independently, or in combination with any one or more of the features described herein.

Unless defined otherwise below, all technical and scientific terms used herein have the same meaning as commonly understood by one of ordinary skill in the art to which this disclosure belongs. Still, certain elements are defined herein for the sake of clarity.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The publications discussed herein are provided solely for their disclosure prior to the filing date of the present application. Nothing herein is to be construed as an admission that the present disclosure is not entitled to antedate such publication by virtue of prior invention. Further, the dates of publication provided can be different from the actual publication dates, which can need to be independently confirmed.

It must be noted that, as used in this specification and the appended claims, the singular forms “a”, “an” and “the” include plural referents unless the context clearly dictates otherwise. Thus, for example, reference to “a biopolymer” can include more than one biopolymer.

### Definitions

The following definitions are provided for specific terms that are used in the following written description.

A “biopolymer” is a polymer of one or more types of repeating units. Biopolymers are typically found in biological systems and particularly include polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions, or in non-Watson-Crick type hydrogen bonding and/or electrostatic interactions (for example, but not limited to, Hoogsten binding and the like). Polynucleotides include single or multiple stranded configurations, where one or more of the strands can or can not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

A “biomonomer” references a single unit, which can be linked with the same or other biomonomers to form a biopolymer (for example, a single nucleotide with two linking groups one or both of which can have removable protecting groups).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally or non naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 200 nucleotides in length, while a “polynucleotide” or “nucleic acid” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties can be covalently or non-covalently bound to the surface in the region. For example, each region can extend into a third dimension in the case where the substrate is porous while not having any substantial third dimension measurement (thickness) in the case where the substrate is non-porous. An array can contain more than ten, more than one hundred, more than one thousand more than ten thousand features, or even more than one hundred thousand features, in an area of less than 20 cm² or even less than 10 cm². For example, features can have widths (that is, diameter, for a round spot) in the range of from about 10 μm to about 1.0 cm. In other embodiments each feature can have a width in the range of about 1.0 μm to about 1.0 mm, such as from about 5.0 μm to about 500 μm, and including from about 10 μm to about 200 μm. Non-round features can have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features can account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature can incidentally detect non-targets of that feature). The target for which each feature is specific is, in representative embodiments, known. An array feature is generally homogenous in composition and concentration and the features can be separated by intervening spaces (although arrays without such separation can be fabricated).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In some embodiments, the collections of oligonucleotide elements employed herein are present on a surface of the same planar support, e.g., in the form of an array. It should be understood that the terms “probe” and “target” are relative terms and that a molecule considered as a probe in certain assays can function as a target in other assays.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

In some embodiments, there are provided herein improved processes for solid phase oligonucleotide synthesis. In these improved processes, the improvement comprises carrying out detritylation of the nascent oligonucleotide having at least one trityl group using a composition comprising a protic acid, a protic solvent, and an organic solvent. In some embodiments of these processes, such synthesis is carried out using the phosphoramidite, H-phosphonate, or phosphotriester approach. In some embodiments, such synthesis may be solid phase synthesis or solution phase synthesis. A “nascent oligonucleotide having one or more trityl group” is intended to include any oligonucleotide in which at least one trityl moiety protects at least one hydroxy functionality. A trityl group can comprise trityl, monomethoxytrityl, dimethoxytrityl, 9-phenylxanthen-9-yl or 9-p-methoxyphenylxanthen-9-yl, for example.

Applicant has observed that the use of certain batches of detritylation reagent obtained commercially caused reduction in the yield of full length of oligonucleotides chemically synthesized on a substrate. The present disclosure is based in part on the surprising discovery by Applicant that adding a protic solvent (such as, for example, water) to the detritylation reagent resulted in the resolution of this problem, thus allowing continued use of the batch of reagent.

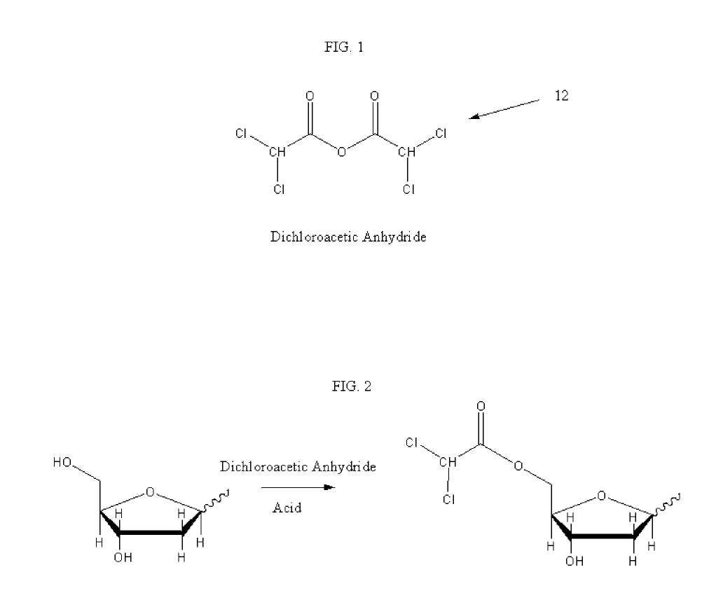

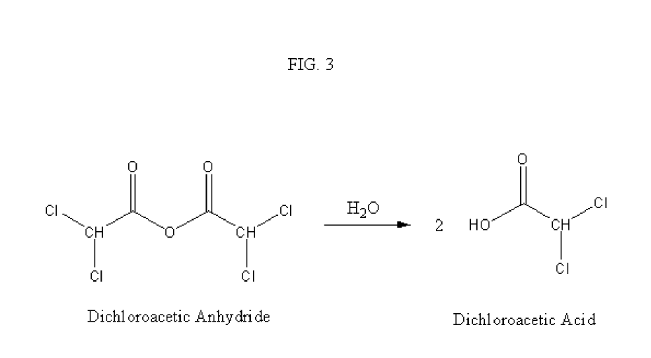

Without wishing to be bound by theory, it is contemplated that an impurity was present in certain batches of ditrytilation reagent that interfered with the chemical synthesis of an oligonucleotide. Is it possible that the protic acid itself is capable of forming such an impurity. FIG. 1 shows an embodiment of such an impurity 12, dichloroacetic anhydride. It is contemplated that when dichloroacetic anhydride is present in sufficient concentration, it acts as a capping agent, as shown in FIG. 2, that recaps the hydroxyl group after the protecting trityl group has been removed by the action of the protic acid detritylation agent. A protic solvent 14 (as exemplified by H₂O in FIG. 3), when present at sufficient concentration, can react with the impurity and thereby essentially eliminate its presence, thus preventing the capping reaction.

A wide variety of protic solvents can be used in the present methods. In some embodiments, the protic solvent can be water, an alcohol, or an amine or mixtures thereof. In some embodiments, the protic solvent comprises at least one of a primary, a secondary and a tertiary alcohol. In some embodiments, the protic solvent comprises at least one of methanol, ethanol, 1-propanol, 2-propanol, 1-butanol and 2-butanol. In some embodiments, the protic solvent comprises at least one of a primary, a secondary and a tertiary amine.

Without wishing to be bound by theory, non-limiting examples of potential impurities which can act as capping agents, and which would be inhibited by the use of a protic solvent as described herein, include ethers (such as, e.g., methoxy methyl ether, tetrahydropyranyl ether, t-butyl ether, etc.), acetic acid derivatives (such as, e.g., esters and anhydrides), 1,3-diols, acetals, pivolates, and benzoates.

The amount of protic solvent that may be utilized during a detritylation step can be determined by routine experimentation, such as by titration, and by varying the reaction time. For example, detritylation reagent can be prepared with various concentrations of one or more protic solvent, and the effect on the detritylation reaction can be monitored over time.

In some embodiments, detritylation of a nascent oligonucleotide having one or more trityl group can be carried out in a composition comprising an organic solvent, a protic acid, and a protic solvent. In some embodiments, a protic solvent is present at a concentration greater than 0.0575% mole fraction. In some embodiments, a protic solvent is present at a concentration greater than 0.115% mole fraction. In some embodiments, the protic solvent is present at a concentration in the range of 0.0575% mole fraction to 1.72% mole fraction, in the range of 0.115% mole fraction to 0.287% mole fraction, or in the range of 0.175% mole fraction to 0.23% mole fraction.

In some embodiments, detritylation of a nascent oligonucleotide having one or more trityl group can be carried out in a composition comprising an organic solvent, a protic acid, and a protic solvent. In some embodiments, a protic solvent is present at a concentration greater than 0.01, 0.02, 0.03, 0.04, 0.05, 0.06, 0.07, 0.08, 0.09 or 0.1% mole fraction. In some embodiments, a protic solvent is present at a concentration greater than 0.1, 0.2, 0.3, 0.4, 0.5, 0.6, 0.7, 0.8, 0.9 or 1.0% mole fraction. In some embodiments, the protic solvent is present at a concentration in the range of 0.05% mole fraction to 2% mole fraction; in the range of 0.05% mole fraction to 5% mole fraction; or in the range of 0.05% mole fraction to 10% mole fraction. In some embodiments, the protic solvent is present at a concentration in the range of 0.1% mole fraction to 0.3% mole fraction.

A composition used in a detritylation reaction as described herein includes a protic acid. There are no particular limits on the protic acid which can be used to effect the detritylation, as long as the protic acid does not interfere with the chemical synthesis of the polynucleotide. Suitable concentrations of protic acid can be determined empirically. The occurrence of side reactions, if observed (such as, for example, depurination), can be monitored, and minimized by titrating the concentration of protic acid and/or utilizing a different protic acid, by varying the reaction time, or by varying the temperature so that the extent of the side reaction is relatively depressed more than the intended reaction.

In some embodiments of the improved processes according to the present disclosure, the detritylation step utilizes a protic acid as a detritylation reagent. As used herein, a protic acid is intended to mean a compound in which hydrogen is attached to oxygen or nitrogen and which has appreciable acidity. In some embodiments, the protic acid is present in the range from 0.625% mole fraction to 25.0% mole fraction. In some embodiments, the protic acid is present in the range from 1.25% mole fraction to 12.5% mole fraction. In some embodiments, the protic acid is present in the range from 0.01% mole fraction to 25.0% mole fraction. In some embodiments, the protic acid is present in the range from 0.1% mole fraction to 12.5% mole fraction. In some embodiments, the protic acid is present at a concentration of 3.75% mole fraction. Non-limiting examples of a protic acid include a halogenoacetic acid, such as, for example, a chloroacetic acid, a bromoacetic acid, or a fluoroacetic acid. For example, a protic acid can comprise at least one of monochloroacetic acid, dichloroacetic acid (DCA) and trichloroacetic acid (TCA). A protic acid can comprise at least one of trifluoroacetic acid, formic acid, sulfuric acid, propanoic acid, para-toluenesulfonic acid and benezenesulfonic acid. In some embodiments, hydrochloric acid can be used.

In some embodiments, the protic acid is present in the range from 0.01% mole fraction to 25.0% mole fraction. In some embodiments, the protic acid is present in the range from 0.1% mole fraction to 12.5% mole fraction.

A wide variety of organic solvents can be used in a composition used in a detritylation reaction as described herein. There are no particular limits on the organic solvent that can be used, as long as it does not interfere with the chemical synthesis of the polynucleotide and as long as the intended reaction can be performed efficiently. In some embodiments, an organic solvent is a liquid hydrocarbon. The organic solvent can comprise, for example, an alkane, a halo-substituted hydrocarbon solvent, a chlorohydrocarbon solvent, or an arene solvent. An organic solvent can comprise at least one of toluene, dichloromethane, dichloroethane, aliphatic substituted benzene, halogenic solvent, 1,2-dichloroethane, and methylene chloride.

In some embodiments, the organic solvent is an alkylbenzene. The aklylbenzene can have a single phenyl ring. Examples of suitable alkylbenzenes include, without limitation, toluene, xylene, hemimellitene, pseudodocumene, mesitylene, prehnitene, isodurene, durene pentamethylbenzene, hexamethylbenzene, ethylbenzene, ethyltoluene, propylbenzene, propyltoluene, butylbenzene, pentanylbenzene, pentanyl toluene, hexanyl benzene and hexanyl toluene. In some embodiments, alkylbenzenes include those having more than one phenyl ring, such as diphenylmethane, triphenylmethane, tetraphenylmethane and 1,2-diphenylethane can be used, examples of which include, without limitation, styrene, stilbene, diphenylethylene, triphenylethylene and tetraphenylethylene. In some embodiments, alkynylbenzenes can be used, and include, without limitation, phenylacetylene and diphenylacetylene.

There are no particular limits on the support used in the chemical synthesis of polynucleotides used in the methods described herein, as long as the support is compatible with the reaction solvents and other reagents utilized in the synthesis. The substrates may be fabricated from a variety of materials. A wide variety of organic polymers or inorganic polymers can be employed (see, e.g., U.S. Pat. Nos. 4,373,071; 4,500,707; 6,171,797; and 6,538,128).

The processes as described herein are useful for synthesizing polynucleotides, including oligonucleotides, on a scale ranging from small laboratory scale to large commercial scale. Thus, the processes can be used to supply oligonucleotides for research purposes, for diagnostic purposes and for therapeutic purposes.

The following examples are intended to further illustrate certain preferred embodiments of the invention and are not intended to be limiting in nature.

### EXAMPLE 1

### Effect of Water Added to a Detritylation Solution

An oligomer of 31 units (SEQ ID NO:1) was synthesized under various conditions using an Applied Biosystems 3400 DNA Synthesizer and analyzed on an Agilent Series 100 HPLC.
SEQ ID NO:1 GTCTCTGCCTTCTTCAGTTTCCTGCTTGCCTT

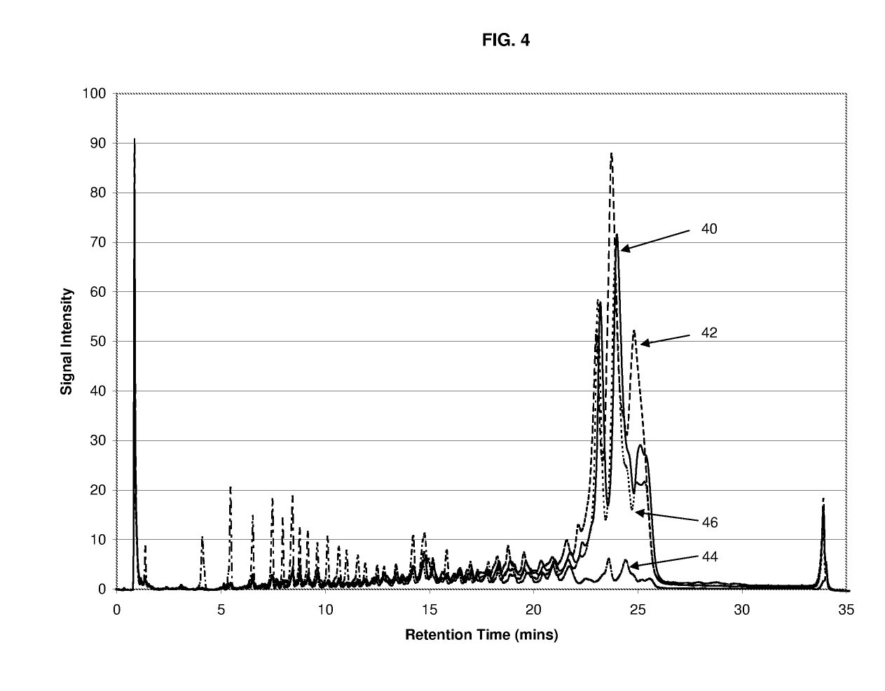

Detritylation solution was obtained from Burdick and Jackson. The detritylation solution contained 3.75% mole fraction dichloroacetic acid (DCA) in toluene (3% DCA vol/vol). The solution contained 0.055% mole fraction of water (95 ppm water). Water content was estimated by Karl Fischer titration using a titrator manufactured by Metrohm, Inc. (Westbury, N.Y.). The duration of the detritylation step was 60 sec. The HPLC trace 40 of the resulting sample is shown in FIG. 4.

In one experiment, water was added to this initial detritylation solution to a final concentration of 0.38% mole fraction (HPCL trace 42).

In another experiment, dichloroacetic anhydride was added to the initial detritylation solution at a concentration of 0.236% mole fraction (0.023 moles/liter), and resulted in a dramatic decrease in the overall yield of high molecular weight oligomer (HPLC trace 44).

In another experiment, a detritylation solution of trichloroacetic acid 3.3% mole fraction (3% vol/vol) in dichloromethane was used (HPCL trace 46).

### Reconstitution of Detritylation Solution Treated with DCAA

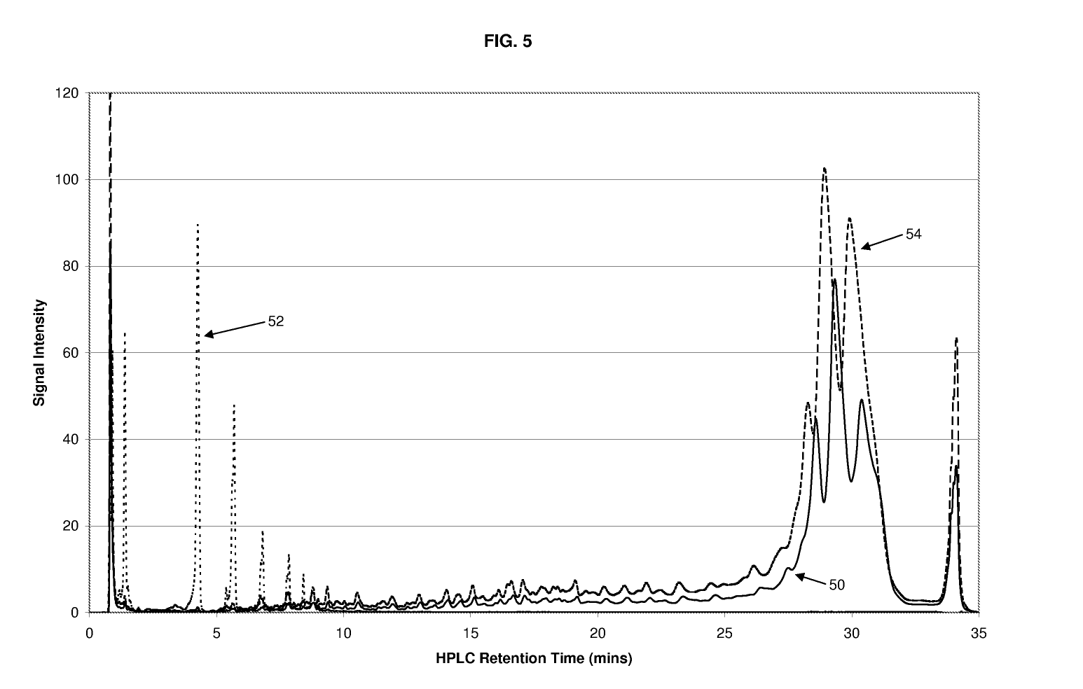

Dichloroacetic anhydride can be cleaved by water to give two molecules of dichloroacetic acid (FIG. 3). FIG. 5 shows that the effects of having an impurity such as dichloroacetic anhydride in the detritylation solution can be ameliorated by the addition of water. This plot shows the HPLC traces from the oligonucleotide synthesized with

a) untreated detritylation solution (trace 50)
b) detritylation solution treated with DCAA (trace 52)
c) the solution from (b) treated again with water (trace 54). It is contemplated that this latter step destroyed any added DCAA and restored the detritylation solution to its original functional condition. These results show that the detritylation solution could be reconstituted, or restored, in this fashion to its former function.

### Neutralization of DCAA by Water

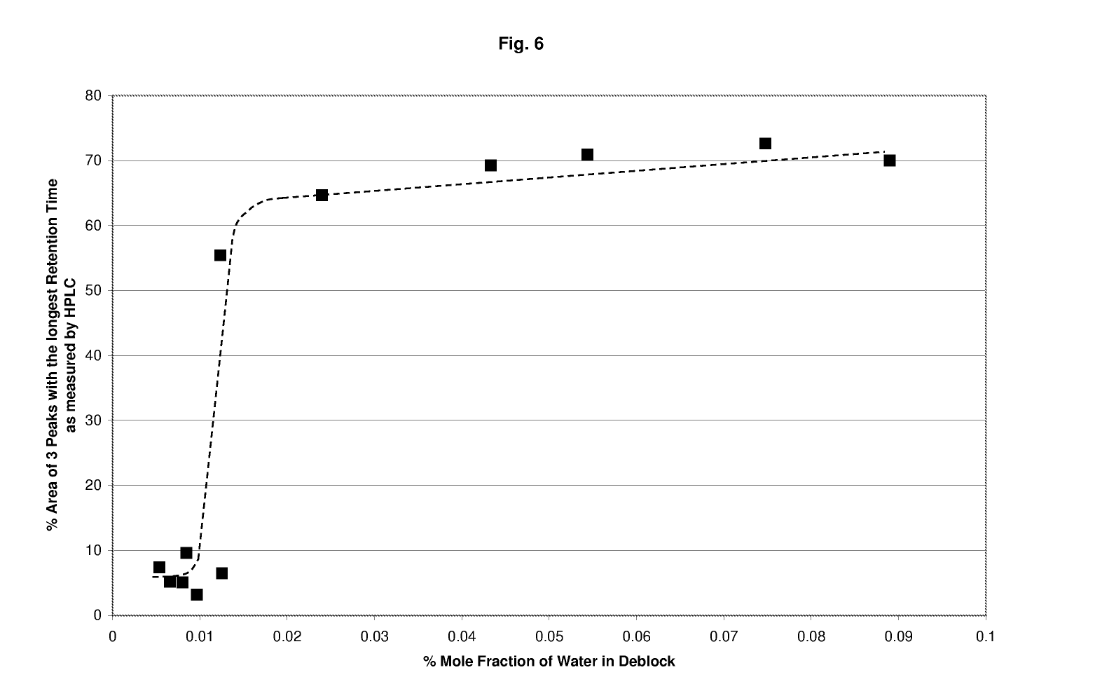

The 31-mer (SEQ ID NO:1) was chemically synthesized but using detritylation solution to which dichloroacetic anhydride had been added as described above. FIG. 6 shows a plot of the percent area, as determined by HPLC of the three peaks with the longest retention times (highest molecular weights) as a function of the concentration of water in the detritylation solution. It can be seen that at a water concentration of about 0.045% mole fraction the synthetic yield started to decline. The results demonstrate the marked sensitivity of the synthetic yield to the water content in the detritylation solution

Those skilled in the art will recognize that many equivalents to the products and processes according to the invention can be made by making insubstantial changes to such products and processes. The following claims are intended to encompass such equivalents.

1. An improved process for solid phase oligonucleotide synthesis, the improvement comprising detritylating a nascent oligonucleotide having one or more trityl group in a composition comprising:
an organic solvent,
a protic acid in the range from 0.625% mole fraction to 25.0% mole fraction, and
a protic solvent present at a concentration greater than 0.0575% mole fraction.

2. The process according to claim 1, wherein the protic solvent comprises at least one of water, alcohols, and amines.

3. The process according to claim 1, wherein the protic solvent comprises water.

4. The process according to claim 1, wherein the protic solvent comprises alcohol.

5. The process according to claim 1, wherein the protic solvent is present at a concentration greater than 0.115 % mole fraction.

6. The process according to claim 1, wherein the protic solvent is present at a concentration in the range of 0.0575% mole fraction to 1.72% mole fraction.

7. The process according to claim 1, wherein the protic solvent is present at a concentration in the range of 0.115% mole fraction to 1.72% mole fraction.

8. The process according to claim 1, wherein the protic solvent is present at a concentration in the range of 0.175% mole fraction to 0.23% mole fraction.

9. The process according to claim 4, wherein the protic solvent comprises at least one of methanol, ethanol, 1-propanol, 2-propanol, 1-butanol and 2-butanol.

10. The process according to claim 1, wherein the protic acid is present at a concentration in the range of 1.25% mole fraction to 12.5% mole fraction.

11. The process according to claim 10, wherein the protic acid is present at a concentration of 3.75% mole fraction.

12. The process according to claim 1, wherein the protic acid comprises a halogenoacetic acid.

13. The process according to claim 12, wherein the protic acid comprises dichloroacetic acid.

14. The process according to claim 12, wherein the protic acid comprises trichloroacetic acid.

15. The process according to claim 1, wherein the protic acid comprises at least one of trifluoroacetic acid, formic acid, propanoic acid, para-toluenesulfonic acid and benezenesulfonic acid.

16. The process according to claim 1, wherein the organic solvent comprises a liquid hydrocarbon.

17. The process according to claim 1, wherein the organic solvent comprises toluene.

18. The process according to claim 1, wherein the organic solvent comprises at least one of toluene, dichloromethane, aliphatic substituted benzene, halogenic solvent, 1,2- dichloroethane.

19. The process according to claim 1, wherein the polynucleotide is attached to an organic polymer support.

20. The process according to claim 1, wherein the polynucleotide is attached to a particle.

21. The process according to claim 1, wherein the polynucleotide is attached to a bead.

22. The process according to claim 1, wherein the polynucleotide is attached to a planar support.

23. An improved process for solid phase polynucleotide synthesis, the improvement comprising detritylating a nascent polynucleotide having one or more trityl group using a composition comprising:
a protic solvent present at a concentration greater than 0.0575% mole fraction.

24. The improved process according to claim 23, wherein the composition comprises a protic solvent present at a concentration greater than 0.115% mole fraction. 25. An improved process for solid phase polynucleotide synthesis, the improvement comprising detritylating the nascent polynucleotide having one or more trityl group in a composition comprising an organic solvent, a protic acid in the range from 0.625to 25.0% mole fraction, and a protic solvent, wherein the protic solvent is present at a concentration sufficient to substantially eliminate an impurity in the composition, the impurity comprising a capping agent.

26. The process according to claim 25, wherein said protic acid is capable of forming said capping agent.

27. A composition for use in a detritylation reaction during chemical synthesis of a polynucleotide, the composition comprising:
an organic solvent,
a protic acid present in the range from 0.625to 25.0% mole fraction, and
a protic solvent present at a concentration greater than 0.115% mole fraction.

28. The composition of claim 27 wherein the protic acid is present at a concentration of about 3.75% mole fraction.

29. The composition of claim 27 wherein the protic solvent comprises water.

30. The composition of claim 27 wherein the protic acid comprises dichloroacetic acid.

31. The composition of claim 27 wherein the protic solvent is present in the range of 0.115% mole fraction to 1.72% mole fraction.
